(19) United States
(12) Patent Application Publication
(10) Pub. No.: US 2003/0176773 A1
(43) Pub. Date: Sep. 18, 2003

(54) Methods of assessing the level of a subjective symptom

(76) Inventors: Roger Michael Gendreau, Poway, CA (US); Srinivas Gandham Rao, San Diego, CA (US)

Correspondence Address: David Spolter, LAW OFFICE OF DAVID SPOLTER, 1590 Coast Walk, La Jolla, CA, 92037, US

(21) Appl. No.: 10/097,184

(22) Filed: Mar. 12, 2002

(51) Int. Cl. G06F 17/60; A61B 5/00

(52) U.S. Cl. 600/300; 705/3

(57) ABSTRACT

The present invention provides methods of assessing the level of a subjective symptom in a human subject. The methods involve randomly prompting the subject for his current level of the subjective symptom. Preferably, the methods are used while the subject is undergoing an intervention to try to decrease or ameliorate the level of the symptom.

### FIELD OF THE INVENTION

The present invention relates to methods of assessing the level of a subjective symptom in a human subject. In particular, the level is assessed while a subject is undergoing an intervention to decrease or ameliorate the level of the symptom.

### BACKGROUND OF THE INVENTION

Assessing the level of a subjective symptom of a human subject accurately over a period of time has long been a problem. Because such a symptom cannot be measured accurately through objective means, traditional assessment necessarily involves asking or “prompting” the subject for the level of the symptom, either at the current moment or recalled over a fixed interval of time.

Indeed, assessment that uses recall has long been the “gold standard” of the U.S. Food and Drug Administration. See, for example New Drug Application 21-107 regarding Lotronex, pages 18-19. Often, the subject is asked to recall the level of the subjective symptom over a past period of time, such as the past week. However, methodologies based on a subject's recall of a subjective symptom are prone to bias due to psychological factors. In particular, a negative affect at the time of the interview or prompt can result in negative events being more easily recalled and, potentially, exaggerated. See J. D. Teasdale & S. J. Fogerty, J. of Abnormal Psych., 88:248-257 (1979); and Schiffman et al., J. of Counseling and Clinic. Psychology, 65:292-300 (1997).

Biased recall has important ramifications when one is trying to assess the effectiveness of an intervention, for example a drug, in decreasing or ameliorating the level of a subjective symptom, for example pain. In particular, pain may be recalled as being more severe in subjects with chronic pain syndromes like fibromyalgia, as such subjects typically also suffer from co-morbid depression or dysthymia. See M. D. Boissevain & G. A. McCain, Pain, 45:239-248 (1991); M. D. Boissevain & G. A. McCain, Pain, 45:227-238 (1991); and J. I. Hudson et al., Am. J. Psychiatry, 142:441-446 (1985).

On the other hand, assessing the current level of a subjective symptom at a specific time of day is also thought to lack sufficient accuracy due to variation in such a symptom throughout the day. Such variation can be due to, for example, diurnal fluctuation or an external factor such as job stress. For example, prompting a subject with rheumatoid arthritis for his current level of stiffness in the morning, when stiffness is typically greatest, may not accurately reflect his average level of stiffness for that day.

Assessing the current level of a subjective symptom at several specific and fixed times of day is also deficient, as the various possible biases are not sufficiently minimized.

In short, improved methods for assessing the subjective symptom of a human subject undergoing an intervention are desired. This invention satisfies this need and provides related advantages as well.

### SUMMARY OF THE INVENTION

The present invention provides methods of assessing the level of a subjective symptom in a human subject. The methods involve randomly prompting the subject for his current level of the subjective symptom. Preferably, the methods are used while the subject is undergoing an intervention to try to decrease or ameliorate the level of the symptom.

### BRIEF DESCRIPTION OF THE DRAWINGS

FIG. 1 shows the compliance rate of subjects who were randomly prompted five to six times per day using a handheld computer over a period of eight weeks.

FIG. 2 shows the comparison of the average values for various assessment methods in all subjects for the first two weeks of the study. For each subject, the first value (from left to right; in medium gray) shows the random prompting assessment, calculated by averaging all responses to random prompts over a two-week period; the second value (in dark gray) shows the average of the daily, non-random, one-day recall prompts over a two-week period; the third value (in light gray) shows the average of two one-week recall assessments; and the fourth value (in black) shows the one-week recall assessment obtained at the clinic visit.

FIG. 3 shows the results of the various methods of assessment for a subject over the entire period of the study. The data from the one-week recall assessment done at clinic visits is represented with triangles. The data from the weekly recall assessments done by the subjects on their handheld computers is represented by small squares. Finally, the data from the one-week averages of the random, current (i.e., non-recall) prompts is represented by diamonds.

### DETAILED DESCRIPTION OF THE INVENTION

The present invention provides methods of assessing the level of a subjective symptom in a human subject. The methods involve randomly prompting the subject for his current level of the subjective symptom. Preferably, the methods are used while the subject is undergoing an intervention to try to decrease or ameliorate the level of the symptom.

As used herein, the term “subjective symptom” means any phenomenon experienced by a subject that cannot be accurately measured using objective means, i.e., by accurate measurement of another or by using an instrument.

Examples of subjective symptoms include pain, anxiety, fatigue, anger, depression, stiffness and sleep quality. More specifically, the pain can be chronic. Even more specifically, the chronic pain can be symptomatic of fibromyalgia syndrome. It should be understood, however, that the present invention can be used to assess the level of any subjective symptom.

As used herein, the term “randomly prompting” means, except for the parameters provided below, having an equivalent probability of a prompt occurring at any given time.

The minimum time between prompts is at least about 15 minutes, but can be about 20 minutes, about 25 minutes, preferably about 30 minutes, and can be about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 16, 16.5, 17, 17.5, 18, 18.5, 19, 19.5, 20, 20.5, 21, 21.5, 22, 22.5, 23, 23.5 or 24 hours.

The average time between prompts is at least about 15 minutes, but can be about 20 minutes, about 25 minutes, preferably about 30 minutes, and can be about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 16, 16.5, 17, 17.5, 18, 18.5, 19, 19.5, 20, 20.5, 21, 21.5, 22, 22.5, 23, 23.5 or 24 hours.

As used herein, the term “intervention” means any action or administration that is intended to decrease or ameliorate the level of a subjective symptom. An example of an intervention is administration of a drug. However, an intervention can also be, for example, an electronic stimulus, psychological therapy, behavioral therapy, acupuncture, light therapy, or relaxation therapy.

As exemplified in Example 2 below, the intervention is administration of milnacipran to decrease the level of chronic pain in subjects with fibromyalgia syndrome.

The present invention provides random prompting occurring as infrequently as once per day, or two, three, four, five, six, seven, eight, nine, ten, eleven, twelve, thirteen, fourteen, fifteen, sixteen or more times per day. Preferably, random prompting occurs four, five or six times per day.

The present invention provides random prompting occurring for one day, or two, three, four, five or six or more days, or one, two or three or more weeks, or one two three, four, five, six, seven, eight, nine, ten, eleven or more months. Preferably, random prompting occurs in two or more consecutive days.

The methods of the present invention include assessing one or more human subjects. Preferably, the methods of assessing include two or more human subjects.

Random prompts can request any quantifiable response, for example, a verbal response (for example, intense, moderate, mild, weak, nothing); a number on a scale; or a continuous response on a visual analog scale (VAS). Preferably, random prompts request a number on a continuous scale. Even more preferably, random prompts request a number on a continuous scale with verbal anchors. An example of such a scale is the Gracely Scale for measuring pain intensity, as described by R. H. Gracely & D. M. Kwilosz, Pain, 35:279-288 (1988).

A scaled response can be adjusted to maximize the effectiveness of a response. For example, a scale can be made logarithmic so that a wider range can be used and each interval represents the same magnitude of change. An example of such a scale is the Gracely Scale, as described above.

Responses to random prompts can be mathematically manipulated to increase their accuracy. For example, responses can be averaged to decrease “noise” or wide variations.

As used herein, the term “mathematically manipulated” means any statistical tool used to make an assessment more amenable to interpretation. Such tools can, for instance, address the issue of high variability in a subject's responses. Examples of such tools include standard deviation, standard error, averaging and the like.

A preferred tool used in the present invention is averaging and, more specifically, “area under the curve” (AUC). As used herein, AUC is an integral of the numerical response data over a period of time. Useful time periods include 1 day, 5 days, 7 days and 14 days.

“Averaging” may take the form of a “moving average,” which, for any given day, entails averaging a past period of time. For example, a 14-day moving average is the average of days 1 to 14, 2 to 15 etc.

Mathematical manipulation can be made for the responses of each day. For example, responses for each day can be averaged to address wide variation in responses for that day. Alternatively, such manipulation can be made for the responses made over a greater period of time, including averaging or integrating the responses from intervals ranging from days, to one or two weeks, to several weeks, to one or two months to several months.

Prompting can be made in any fashion. Similarly, responses can be made in any fashion. Preferably, prompting and responses are made through an electronic device, for example a Palm Pilot or any other handheld computer. See Shiffman et al., supra., which discusses use of such a device.

The present invention further provides prompting a subject before an intervention is made. Such prompting can occur for one or more days, two, three four, five or six days or more days, or one, two or three or more weeks, or one two three, four, five, six, seven, eight, nine, ten, eleven or more months. Preferably, such prompting occurs for five or more days, or one to two weeks. Such prompting may be random. Such responses may be mathematically manipulated, as discussed above.

The present invention further provides prompting a subject for his non-current level of a subjective symptom over a past period of time in conjunction with prompting for the current level. Typical past periods are the previous day, previous week and previous month. Typically, the subject responds with the average level of the subject symptom over the relevant period of time.

The invention also provides non-random prompting in conjunction with random prompting. Typical non-random times are morning and evening.

The invention further provides recall prompting in conjunction with current, random prompting. Typical recall prompting periods include the past day, week, two weeks, month, two months and three months.

A response can occur at any time after a random prompt is made. Preferably, a response is made before the next prompt occurs. More preferably, a response is made within one hour of a prompt. Even more preferably, a response is made with about 30 or 35 minutes after a prompt. Most preferably, a response is made within about five minutes of a prompt.

The following examples are provided to illustrate but not limit the present invention.

### EXAMPLE 1

This example shows that random prompting of human subjects can more accurately assess their level of a subjective symptom such as pain.

Fourteen human subjects diagnosed with primary fibromyalgia were enrolled in a study over a three-month period to assess their level of pain. For the diagnostic criteria for primary fibromyalgia, or fibromyalgia syndrome, see for example Reid et al., British Medical Journal, 320:292-296 (2000).

An electronic diary was provided to each subject. The diary made four to six random prompts the diary to each subject daily. Each prompt asked the subject to rate his current level of pain.

To set the prompts, a randomization algorithm was used so that such prompts for each subject would not occur within 30 minutes of each other.

In addition, other pain assessment tools were used so that they could be compared. Specifically, a daily pain recall prompt was made each morning at a fixed time, asking for assessment of the subject's average pain for the previous day.

Additionally, a weekly recall prompt was made each Friday evening at a fixed time. This prompt asked for assessment of the subject's average pain for the previous week. Finally, a monthly pain recall assessment was made during a subject's clinic visit, asking for assessment of the subject's average pain for the previous month.

In assessing their pain, subjects used the Gracely Scale, which is described above. The scale was logarithmic so that a wider range of the scale was used. The scale had verbal anchors added to describe the level of pain so that responses were more consistent. Consistent responses were promoted through the use of verbal anchors that assisted the subjects in placing their level of pain experience on the scale guided by specific descriptors with face validity. Specifically, the verbal anchors added in the order of greater to lesser was as follows: “extremely intense,” “very intense,” “intense,” “strong,” “slightly intense,” “barely strong,” “moderate,” “mild,” “very mild,” “weak,” “very weak,” “faint” and “no pain sensation.”

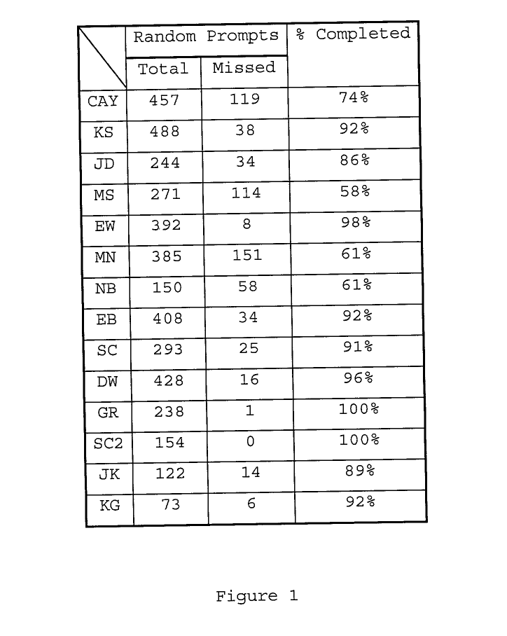

Over the three-month period of the study, response to random prompts was good, ranging from 58% to 100%, with 10 of the 14 subjects studied complying 86% or more. See FIG. 1.

For the first two weeks of the study, a score was calculated for each method of assessment. Specifically, responses to random prompts for the first two weeks (about 60 prompts) were averaged for each subject. This value was computed by averaging the responses to prompts for each week. Thus, equal weight was placed on each of the first two weeks, even if the number of random prompts for each week differed.

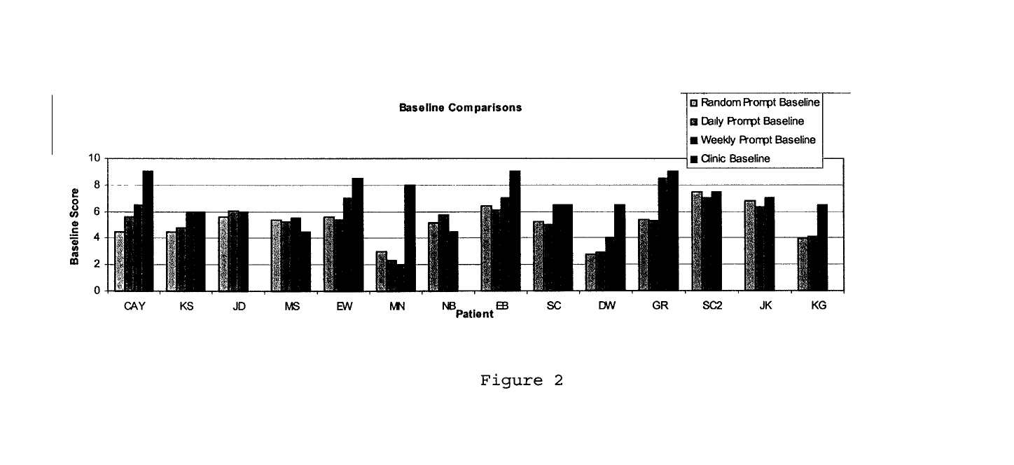

Daily recall responses for the first two weeks (14 prompts) were also averaged for each subject. In addition, a weekly recall response for the first two weeks (2 responses) was averaged for each subject. Finally, a weekly recall report made at the initial clinic visit was also recorded. A comparison of these average responses is shown in FIG. 2.

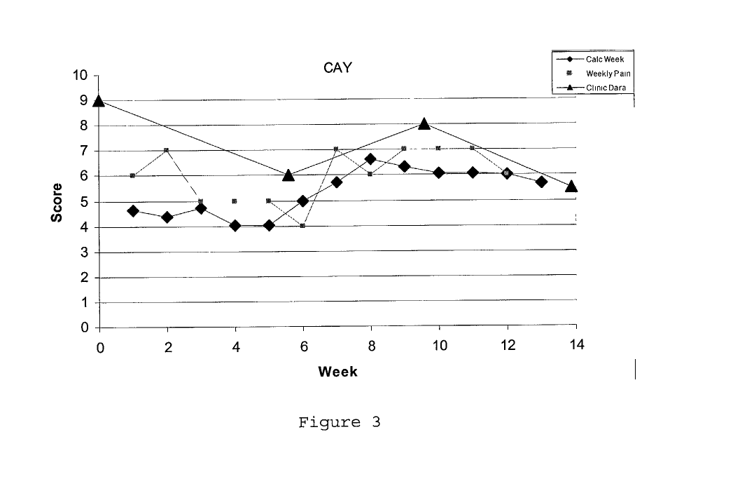

As shown in FIG. 3, which shows the results for one of the subjects in the study using the various forms of assessment, the first two week results using non-random methods appear exaggerated compared to subsequent results, especially when using the weekly recall assessment in the clinic. By contrast, the random prompt assessment did not have this exaggeration.

Generally, an exaggeration during the initial period of a study using recall assessment may be due to a subject's exaggerating through recall to justify being in the study, as well as the other factors described above. A subsequent decrease after this initial phase (without treatment or even a placebo) may be due to “hand holding” and comfort once a subject is in the study a sufficient period of time.

In addition, many of the other assessments, especially weekly recall, appear to be high throughout compared to the random prompting assessments.

Accordingly, these results show that random prompting for current pain, rather than a recalled value, is a more accurate method of assessment.

### EXAMPLE 2

This example shows that random prompting of human subjects can more accurately assess their level of a subjective symptom such as pain while undergoing an intervention such as administration of a drug such as milnacipran to try to decrease or ameliorate the level of the symptom.

As described in Example 1, a baseline was calculated for subjects responding to random prompts, as well as using the other methods for assessing pain as described above.

Subjects are then given a trial of a drug, such as milnacipran, to try to reduce their level of pain. Dosage can be about 100 mg po bid, but may be altered. The duration of use could be at least about four weeks and, preferably, about six to about eight weeks.

In addition to the group receiving a dosage, a placebo group is used, with the results of both groups compared.

In comparing the various methods of assessment for the group receiving the drug, random prompting should more accurately show whether the drug is effective because, as discussed in Example 1, the (pre-dosage) baseline values will not be exaggeratedly high. By contrast, the other methods of assessment will make judging the effectiveness of the drug more difficult, because it will be unclear whether an improvement in pain scores is due to the drug or merely a comparison to an exaggeratedly high baseline.

Moreover, for the reasons discussed herein, responses to random prompting are more accurate all along (both pre- and post-drug administration) than are the responses using the other methods.

Similarly, in comparing the treated and placebo groups using the random prompting assessment, it becomes much clearer to what extent the placebo has an effect as compared to the drug. By contrast, using the other methods of assessment, it is less clear what the extent of these effects are due to the possibility of an exaggeratedly high baseline.

Accordingly, this example shows the usefulness and relative accuracy of a method of assessing the level of a subjective symptom, such as pain, in an interventional trial.

Each published reference cited in this specification is hereby incorporated by reference in its entirety.

Although the invention has been described with reference to the examples provided above, it should be understood that various modifications can be made by those skilled in the art without departing from the invention. Accordingly, the invention is set out in the following claims.

We claim:

1. A method of assessing the level of a subjective symptom in one or more human subjects, comprising randomly prompting said subject for his current level of said symptom while undergoing an intervention to decrease or ameliorate the level of said symptom.

2. The method of claim 1, where said subject is prompted four or more times per day.

3. The method of claim 1, where said subject is prompted for two or more consecutive days.

4. The method of claim 1, comprising randomly prompting two or more subjects.

5. The method of claim 1, where said intervention comprises administration of a drug to said subject.

6. The method of claim 1, where said symptom is selected from the group consisting of pain, anxiety, fatigue, depression, anger, sleep quality and stiffness.

7. The method of claim 6, where said symptom is pain.

8. The method of claim 7, where said pain is chronic.

9. The method of claim 8, where said subject has been diagnosed with fibromyalgia syndrome.

10. The method of claim 1, where said subject responds with a number on a scale.

11. The method of claim 10, where said scale has verbal anchors.

12. The method of claim 10, where said scale is logarithmic.

13. The method of claim 10, where the response of said subject is mathematically manipulated.

14. The method of claim 7, where said subject responds using the Gracely Scale.

15. The method of claim 1, where said prompt is electronic.

16. The method of claim 1, where said patient responds to said prompt electronically.

17. The method of claim 1, further comprising non-randomly prompting said subject for his level of said symptom.

18. The method of claim 17, where the response of said subject to said non-random prompt is his non-current level of said symptom.

19. The method of claim 1, further comprising prompting said subject prior to said intervention.

20. The method of claim 19, where said prompt prior to said intervention is random.

21. The method of claim 19, where said subject is prompted over a period of five or more days prior to said intervention.

22. The method of claim 19, where said subject is prompted over a period of fourteen or more days prior to said intervention.

23. The method of claim 19, where said subject is prompted prior to said invention two or more times per day.

24. The method of claim 23, where the responses of said subject are mathematically manipulated.
